(19) United States
(12) Patent Application Publication
(10) Pub. No.: US 2024/0197153 A1
(43) Pub. Date: Jun. 20, 2024

(54) DEVICE AND METHOD FOR COVERING AN ENDOSCOPE WITH A SLEEVE AND/OR FOR COUPLING AN ENDOSCOPE WITH ONE OR MORE ADD-ON TUBES

(71) Applicant: Motus GI Medical Technologies Ltd., Tirat HaCarmel (IL)

(72) Inventors: Iddo SALMAN, Tirat Carmel (IL); Rephael SOMMER, Nesher (IL); Mark POMERANZ, Leesburg, VA (US); Jaacob GILBOA, Tel Adashim (IL)

(73) Assignee: Motus GI Medical Technologies Ltd., Tirat HaCarmel (IL)

(21) Appl. No.: 18/287,049

(22) PCT Filed: Jun. 2, 2022
(86) PCT No.: PCT/IL2022/050588

§ 371 Date: Oct. 16, 2023

Related U.S. Application Data
(60) Provisional application No. 63/195,749, filed on Jun. 2, 2021.

(51) Int. Cl. A61B 1/00

(52) U.S. Cl. CPC A61B 1/00135; A61B 1/00066

(57) ABSTRACT

A handle for at least partially covering an endoscope with a protective thin sleeve, the handle comprising: a handle body shaped and sized to receive a distal portion of an endoscope therein; a detachable portion at a proximal end of the handle body, the detachable portion configured to detach from the handle body and to be moved proximally along at least a portion of a length of an endoscope received within the handle body; and a thin elongate protective sleeve axially compacted within the handle body, a proximal end of the sleeve coupled to the detachable portion, wherein movement of the detachable portion proximally along the endoscope pulls on the proximal end of the sleeve and extends the sleeve in length from its axially compacted state to a lengthened state, thereby covering the at least a portion of the length of the endoscope.

### RELATED APPLICATION

This application claims the benefit of priority of U.S. Provisional Patent Application No. 63/195,749 filed on 2 Jun. 2021, the contents of which are incorporated herein by reference in their entirety.

### FIELD AND BACKGROUND OF THE INVENTION

The present invention, in some embodiments thereof, relates to a covering an endoscope with a protective sleeve and, more particularly, but not exclusively, to a handle for facilitating dressing of a sleeve on an endoscope and/or for coupling an endoscope to an add-on tube.

US Publication number US20060111611A1 discloses “Apparatus (20) for sheathing an endoscope (22) includes a dispenser (24), having entry and exit ports (36, 39) defining a transit passage through which the endoscope may pass, and a flexible sleeve (26), at least a portion of which is bunched in a vicinity of the dispenser. The sleeve includes a distal end (28), which is closed, and a proximal end, which is open and fixed to the dispenser so that as the endoscope is advanced in a distal direction through the transit passage, the endoscope enters into the sleeve through the proximal end and engages the distal end of the sleeve, thus causing the bunched portion of the sleeve to be extended so as to cover a distal part of the endoscope that protrudes through the exit port.”

### SUMMARY OF THE INVENTION

According to an aspect of some embodiments there is provided a handle for at least partially covering an endoscope with a protective thin sleeve, the handle comprising:
a handle body shaped and sized to receive a distal portion of an endoscope therein;
a detachable portion at a proximal end of the handle body, the detachable portion configured to detach from the handle body and to be moved proximally along at least a portion of a length of an endoscope received within the handle body; and
a thin elongate protective sleeve axially compacted within the handle body, a proximal end of the sleeve coupled to the detachable portion, wherein movement of the detachable portion proximally along the endoscope pulls on the proximal end of the sleeve and extends the sleeve in length from its axially compacted state to a lengthened state, thereby covering the at least a portion of the length of the endoscope.

In some embodiments, the detachable portion comprises a ring which is separable from the handle body, the ring sized to circumferentially surround an endoscope received within the handle body.

In some embodiments, the handle body accommodates an expandable head sized to receive a distal end of the endoscope therein, the expandable head coupled to a distal end of the sleeve.

In some embodiments, the expandable head is coupled to a distal end of an add-on tube to be inserted along with the endoscope into the body, the expandable head configured to hold a distal end of the endoscope together with the distal end of the add-on tube.

In some embodiments, the handle further comprises a plunger positioned distally to the expandable head, the plunger comprising arm extensions shaped and configured to fit into the expandable head to widen the expandable head for receipt of an endoscope distal end therein.

In some embodiments, the expandable head is elastically expanded to a diameter of between 10-20 mm to fit endoscopes having a diameter of between 9-15 mm.

In some embodiments, the handle body comprises an external radially outwardly extending flange at an axial positon which is intermediate the plunger and the expandable head.

In some embodiments, the detachable portion further comprises a gripper which is separable from the handle body, the gripper circumferentially surrounding the add-on tube and an endoscope received within the handle, the gripper configured to slide proximally to align and approximate the add-on tube to the endoscope.

In some embodiments, the sleeve is formed of a flexible material.

In some embodiments, the sleeve is transparent at least at a distal end face of the sleeve. According to an aspect of some embodiments there is provided a method of covering an endoscope with a protective thin sleeve, comprising:
providing a loading handle containing an axially-collected protective thin sleeve therein;
inserting a distal end portion of an endoscope into a lumen of the loading handle such that the distal end portion of the endoscope is received within the sleeve;
detaching a proximal portion of the handle which is coupled to a proximal end of the sleeve; and
pulling the proximal portion of the handle along a length of the endoscope to cover the endoscope with the sleeve.

In some embodiments, the method further comprises, following an endoscopy procedure, removing the sleeve from the endoscope by pulling the proximal portion of the handle in a proximal direction along the length of the endoscope, thereby rolling up the sleeve.

In some embodiments, the method comprises coupling, at the handle, a distal end of an add-on tube to the distal end portion of the endoscope; wherein the pulling comprises pulling the proximal portion of the handle over the add-on tube and the endoscope to cover them together by the sleeve.

In some embodiments, coupling comprises positioning the distal end portion of the endoscope within a coupling element accommodated inside the lumen of the handle, the coupling element attached to the distal end of the add-on tube.

In some embodiments, pulling of the proximal portion of the handle is configured to align and approximate the add-tube to the endoscope along their length.

In some embodiments, the method comprises disposing the handle, sleeve and add on tube following use.

In some embodiments, the endoscope comprises a colonoscope.

In some embodiments, the endoscope comprises a colonoscope and the add-on tube is suitable for suction and/or irrigation.

According to an aspect of some embodiments there is provided a handle for aligning an endoscope and an add-on tube, the handle comprising:
a handle body which accommodates a coupling element attached to distal end of an add-on tube;
a detachable portion at a proximal end of the handle body, the detachable portion configured to slide proximally along at least a segment of a length of the add-on tube and of an endoscope at least partially received within the handle body to align the add-on tube with the endoscope along their length and approximate them to each other.

In some embodiments, the detachable portion comprises a gripper sized to circumferentially surround the add-on tube and the endoscope.

According to an aspect of some embodiments there is provided a handle for spirally mounting an add-on tube on an endoscope, the handle comprising: a frame comprising:
a recess in which at least a portion of an endoscope is received;
a cylindrical portion on which an add-on tube is axially rolled;
at least a portion of the frame configured to slide along a length of the endoscope and release the rolled add-on tube such that the add-on tube is spirally disposed about the endoscope.

According to an aspect of some embodiments there is provided a sleeve for covering an endoscope, the sleeve comprising:
an elongate sheath having a closed distal end, the sheath extendible from an axially collected form to an elongate, stretched form; and
a gripping element coupled to a proximal end of the elongate sheath and configured to pull on the sheath, when being pulled on by a user, to change a form of the sheath from the axially collected form to the elongate stretched form.

In some embodiments, the gripping element is shaped as a ring having a diameter large enough to circumferentially surround an endoscope.

According to an aspect of some embodiments there is provided a head for coupling a distal end of an add-on tube to a distal end of an endoscope, the head comprising:
a plurality of portions shaped to define, when attached to each other, an inner lumen shaped and sized for fittingly receiving the endoscope distal end therein, the plurality of portions configured to interlock with each other; and
a pocket shaped and sized for receiving the add-on tube distal end therein, the pocket being externally mounted on at least one of the plurality of portions and located radially outwardly relative to the inner lumen.

In some embodiments, the plurality of portions attach to each other via a snap-fit interface.

According to an aspect of some embodiments there is provided a head assembly for use with an endoscope, the head assembly comprising:
an expandable head for mounting onto a distal end of an endoscope, the head comprising a circumferential wall which defines a inner recess, the circumferential wall being expandable in a radially outwards direction;
a plunger shaped to compressively fit inside at least a portion of the inner recess to thereby push the circumferential wall radially outwardly to provide for insertion of the endoscope distal end into the inner recess.

In some embodiments, the plunger comprises a plurality of arm extensions configured for insertion into the inner recess, wherein the arm extensions are pushed radially outwardly against the circumferential wall of the head when the plunger is advanced into the inner recess.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a general method for covering an endoscope along with an add-on tube by an elongate thin sleeve prior to insertion into the body, according to some embodiments;

FIG. 2A schematically illustrates a loading handle pre-attached to an add-on tube and encasing a thin elongate sleeve for covering an endoscope and the add-on tube, according to some embodiments;

FIG. 2B shows a loading handle pre-attached to an add-on tube and encasing a thin elongate sleeve for covering an endoscope and the add-on tube, according to some embodiments;

FIG. 3 is flowchart of a detailed method for covering an endoscope along with an add-on tube by an elongate thin sleeve using the loading handle of FIG. 2B, according to some embodiments;

FIGS. 4A-J show the process of covering an endoscope and an add-on tube by an elongate thin sleeve using a loading handle, according to some embodiments;

FIG. 5 is a flowchart of a method for removing the thin elongate sleeve and separating the add-on tube from the endoscope following the endoscopic procedure, according to some embodiments;

FIGS. 6A-D show the process of removing the thin elongate sleeve and detaching the add-on tube from the endoscope, according to some embodiments;

FIGS. 7A-D are various views of a loading handle, according to some embodiments;

FIGS. 8A-C are various views of the plunger and the inner proximal portion of the loading handle which includes a coupling element (e.g. an expandable head) for coupling the add-on tube to the endoscope, according to some embodiments;

FIG. 9 is an image of a coupling element for coupling a distal end of an endoscope to a distal end of an add-on tube, according to some embodiments.

FIGS. 10A-C show a handle for spirally coupling an add-on tube to an endoscope (FIG. 10A), and schematic views of an add-on tube spirally coupled to an endoscope (FIGS. 10B-C), according to some embodiments;

FIGS. 11A-B show an example of a partial, non-uniform sleeve arranged along an endoscope and an add-on tube, according to some embodiments;

FIG. 12 shows a sleeve coupled to a pullable element (e.g. a handle or part of a handle) for dressing the sleeve on an endoscope according to some embodiments; and

FIGS. 13A-B show a snap-fit head for coupling an endoscope to an add-on tube and for dressing a sleeve on the endoscope and add-on tube, according to some embodiments.

### DESCRIPTION OF SPECIFIC EMBODIMENTS OF THE INVENTION

The present invention, in some embodiments thereof, relates to a covering an endoscope with a protective sleeve and, more particularly, but not exclusively, to a handle for facilitating dressing of a sleeve on an endoscope and/or for coupling an endoscope to one or more add-on tubes.

An aspect of some embodiments relates to a handle for dressing an endoscope, for example, a colonoscope or a gastroscope with a thin elongate sleeve that protects the endoscope, where the sleeve is pulled along the length of the endoscope using a detachable portion of the handle.

In some embodiments, the sleeve is encased within the handle, optionally in a compacted, e.g. axially-collected state. In use, a distal end portion of an endoscope is inserted into a lumen defined by the housing of the handle, and is received inside the compacted sleeve. For covering the endoscope along its length with the sleeve, a proximal detachable portion of the handle which is coupled to a proximal end of the sleeve is pulled in a proximal direction, pulling the sleeve over the endoscope and extending the sleeve from its axially-collected state into a lengthened state in which the sleeve is dressed along at least a portion of the endoscope length (for example, a portion intended for insertion into the body).

In some embodiments, following use of the covered endoscope, the sleeve is removed from the endoscope for example by pulling the detachable handle portion back distally, which pulls on a proximal end of the sleeve to roll it back up, until the sleeve is removed from a distal end of the endoscope.

Some potential advantages of a manually operated handle for facilitating dressing and/or removal of a sleeve onto an endoscope may include: that the process can be carried by a single user (e.g. physician, nurse, technician or other); that no prior inflation of the sleeve is required, avoiding the need for machinery, electrical parts or the like which may be required in processes involving inflation of the sleeve prior to insertion of the endoscope.

A potential advantage of dressing the sleeve onto the endoscope by pulling the sleeve over the endoscope (as opposed to, for example, advancing the endoscope into a lumen of the sleeve) may include reducing or avoiding tearing of the sleeve during covering, for example since the endoscope is not pushed against the walls of the sleeve, but rather remains stationary while the sleeve is pulled over its length.

In some embodiments, no handle is provided, and the sleeve is dressed onto an endoscope using a pulling element such as a ring or tab which is optionally pre-attached to the sleeve proximal end. Optionally, in such configuration, the sleeve includes a closed distal end, so that in use, a distal head of an endoscope can be pushed against the closed end of the sleeve, while the pulling element is moved in a proximal direction along the length of the endoscope, thereby stretching the sleeve along the endoscope.

An aspect of some embodiments relates to coupling an endoscope (e.g. a colonoscope, a gastroscope, or any other elongate medical device configured for insertion into the body) to an add-on tube, and optionally covering the endoscope and add-on tube together by a protecting thin sleeve. In some embodiments, using a handle for example as described herein, the add-on tube and endoscope can be held together at their distal ends, approximated to each other and/or aligned with respect to each other. In some embodiments, approximating and optionally aligning the add-on tube and the endoscope is carried out by moving (e.g. sliding by pulling) a detachable proximal portion of the handle which encompasses both the add-on tube and the endoscope.

In some embodiments, distal ends of the add-on tube and the endoscope are held together and maintained attached by a coupling element which is optionally accommodated inside the lumen of the handle. In some embodiments, the coupling element is adjustable in size, for example in diameter, so that it can selectively fit endoscopes of various diameters. In an example, the coupling element is comprised of an expandable head, which can be widened for example by applying a pushing force on an inner side of the walls of the head. In an exemplary construction, the pushing force is applied by a plurality of arm extensions of a plunger which is advanceable into a lumen of the handle.

In some embodiments, a plurality of add-on tubes (e.g. 2, 3, 4, 5, 7 or intermediate, larger or smaller number of add-on tubes) are coupled to the endoscope. Optionally, in such configuration, the coupling element (e.g. expandable head) is shaped and configured to accommodate the distal ends of the plurality of add-on tubes, and to hold them together with the endoscope distal head.

In some embodiments, an add-on tube comprises a single elongate lumen. Alternatively, an add-on tube is divided into multiple inner lumens, for example, 2, 3, 4, 5, 8, 10 or intermediate, larger or smaller number of lumens. Optionally, the multiple inner lumens are formed using elongate partitions extending along the length of the add-on tube. In some embodiments, in use, the multiple lumens may serve for different functions, for example, one lumen for irrigation and another for suction, and as such.

In some embodiments, a handle for example as described herein is a dual function handle configured for both holding an add-on tube to an endoscope, and covering both along their length with a protective sleeve. Alternatively, in some embodiments, the handle serves only for covering an endoscope with a sleeve. Alternatively, in some embodiments, the handle serves only for coupling and/or aligning an add-on tube with an endoscope.

In some embodiments, the handle is configured to axially align the add-on tube relative to the endoscope, such that the endoscope and add-on tube are parallel to each other. Alternatively, in some embodiments, the handle is configured to dispose the add-tube in a non-parallel orientation, for example, pulling of the handle along the length of the endoscope spirally disposes the add-on tube about the endoscope. Optionally, in such configuration, an add-tube is pre-rolled onto a body or frame of the handle, and pulling of the handle relative to the endoscope gradually releases the rolled add-tube in a spiral orientation around the endoscope.

As referred to herein, the term “proximal” refers to a direction which is one or more of: away from the body of the patient, towards the user (e.g. physician), towards a proximal handle of the endoscope, towards an operating console of the endoscope, and the like. The term “distal” refers to a direction which is one or more of: towards the body of the patient, towards device portions which are inserted into the body (e.g. an examining (e.g. camera) head of an endoscope).

It is noted that in some embodiments, a loading handle for example as described herein may be provided with only some of the described components, optionally, set per the need: for example, for coupling an add-on tube to an endoscope without dressing a sleeve, the handle may be provided without a sleeve contained inside it; for example, for covering an endoscope with a sleeve without coupling the sleeve to an add-on tube the handle may be provided without an expandable head (in which case the sleeve may have a closed distal end); for example, a handle may be provided without a handle body, including for example only a head portion and a sliding portion (e.g. ring or gripper), etc.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

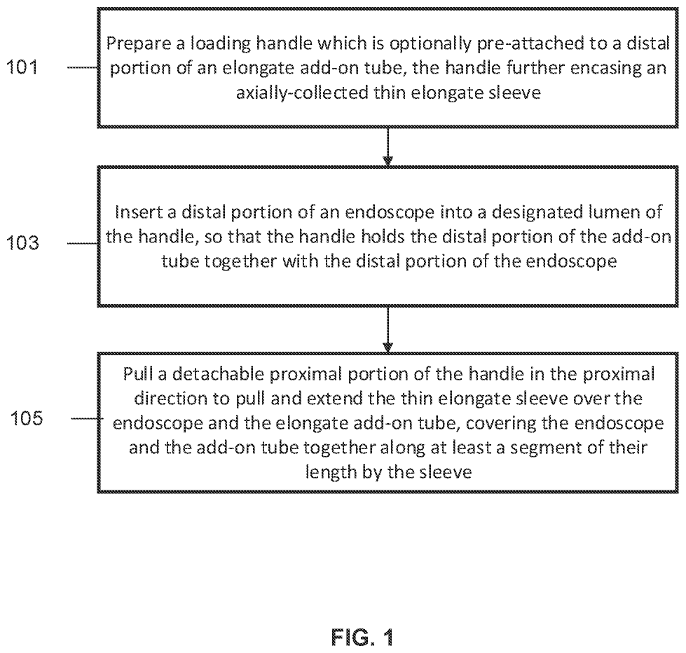

Referring now to the drawings, FIG. 1 is a flowchart of a general method for covering an endoscope along with an add-on tube by an elongate thin sleeve prior to insertion into the body, according to some embodiments.

In some embodiments, prior to a procedure in which an endoscope is inserted into the body (e.g. endoscopy), it is desired to cover the endoscope with a thin sleeve, for example with a single-use, disposable sleeve.

In some embodiments, the sleeve is removed from the endoscope following use, and optionally disposed of.

In some cases, during use, if an endoscope is required to be removed from the body and then re-inserted back into the body, the sleeve may be removed from the endoscope as needed and then dressed back on the endoscope as needed.

At 101, an operator (e.g. physician, nurse, technician or other clinical personnel) prepares a loading handle for engagement with an endoscope. In some embodiments, preparing the handle is by moving at least a portion of the handle, for example, by pulling, rotating, pushing or otherwise moving a portion of the handle to place the handle in a ready-to-load state. In an example, the handle is cocked (e.g. by pulling distally a plunger of the handle) prior to inserting of the endoscope into the handle lumen.

In some embodiments, the handle is pre-attached to or encases a distal end portion of an add-on tube, which is intended to be aligned alongside the endoscope, and optionally covered along with the endoscope by a same single sleeve. In an example, the endoscope is a colonoscope and the add-on tube is insertable along with the colonoscope into the colon, for example for providing suction and/or irrigation and/or imaging, and/or used as an additional working channel or biopsy collection channel. Alternatively, in some embodiments, the handle does not include (for example, is not pre-attached to) an add-on tube. Optionally, the handle is used solely for dressing the thin elongate sleeve on the endoscope.

In some embodiments, the handle encases the thin elongate sleeve, which is optionally axially-collected or otherwise folded or compacted within a housing of the handle.

In some embodiments, a sleeve is selected with properties suitable for the endoscope being used, for example taking into consideration a diameter of the endoscope, a length of the endoscope, a rigidity of the endoscope.

In some embodiments, the sleeve is flexible, for example made of a thin plastic material such as polyurethane (PU), cPTFE, Polyethylene (PE), TPE, TPU. In some embodiments, the sleeve is elastic. In some embodiments, a thickness of the sleeve is lower than 100 μm, lower than 70 μm, lower than 120 μm or intermediate, higher or lower thickness. In some embodiments, the sleeve is shaped and sized to cover at least an endoscope segment which is inserted into the body, for example, having a diameter and/or length sized to fit the endoscope. In some embodiments, the sleeve is cylindrical, having a circular cross section profile. Alternatively, in some embodiments, the sleeve is otherwise shaped, for example is comprised of a sheath which is rectangular in cross section, and is optionally fastened to the endoscope. In some embodiments, in the extended state, the sleeve is long enough to cover an endoscope from a distal end thereof to a proximal end or proximal handle portion of the endoscope, for example, a length of the sleeve is between 100-170 cm, 70-200 cm, 90-150 cm or intermediate, longer or shorter. In some embodiments, sleeves of various lengths for fitting endoscopes of various lengths are provided.

In some embodiments, a distal end face of the sleeve is clear and transparent so that it can be used to cover a distal end face of the endoscope, thereby potentially providing coverage of the endoscope as a whole, without portions of the endoscope being exposed. In some embodiments, the transparent distal end face of the sleeve enables visualization using optical means (e.g. a camera) of the endoscope distal end.

Alternatively, the sleeve is open at its distal end face.

In some embodiments, the sleeve is transparent along its whole length.

In some embodiments, the sleeve is formed of a uniform material, e.g. fully formed of a thin transparent plastic material. A potential advantage of a uniform sleeve may include fully protecting the endoscope from the surroundings, such as from fecal matter, bodily fluids and the like

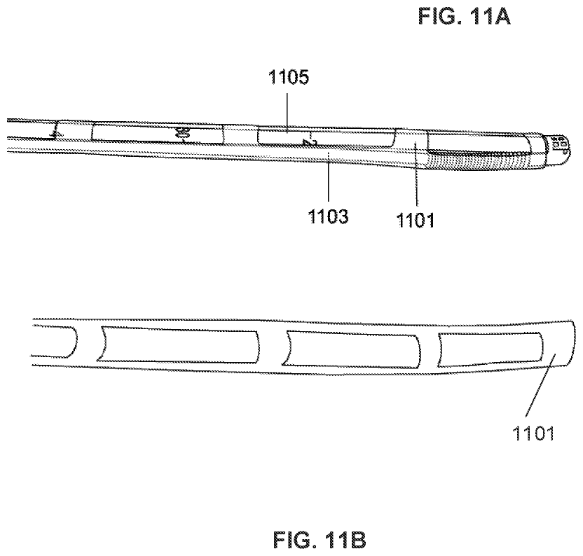

Alternatively, the sleeve is non-uniform, for example comprising openings or portions that do not include material. For example, the sleeve comprises a mesh, multiple elongate stripes, and/or other non-uniform profiles. In an example, the sleeve is formed as a mesh including apertures having, for example, rounded or rectangular cross section profiles. A potential advantage of a mesh sleeve may include obtaining an improved grip of the endoscope and add-on tube, and may potentially reduce interference of the sleeve with bending of the endoscope. Another potential advantage of a mesh sleeve may include a reduced contact surface with the inner walls of the GI tract, for example as compared to a sleeve that is uniform, for example, has no radially outward facing apertures. An example of a non-uniform sleeve is shown in FIGS. 11A-B, showing a sleeve 1101 that circumferentially surrounds an add on tube 1103 and an endoscope 1105 only at a plurality of spaced apart locations along their length, leaving intermediate portions of the add-on tube and the endoscope exposed. Sleeve 1101 is shown separately in FIG. 11B.

In some embodiments, the non-uniform sleeve is comprised of a sheath which has multiple cut-out portions, for example, rectangular openings. In some embodiments, the non-uniform sleeve is dressed on the endoscope by pulling on a proximal end of the sleeve (such as using a pulling element, e.g. a ring coupled to a proximal end of the sleeve).

In some embodiments, the sleeve is at least partially expandable in diameter. Optionally, the sleeve is configured to closely adhere to endoscopes of various diameters. In some embodiments, the sleeve is resilient. In some embodiments, the sleeve widens radially outwardly when being pulled onto the endoscope to fittingly engage the endoscope.

In some embodiments, no sleeve is provided. Optionally, the handle is used for aligning and/or coupling the endoscope to an add-on tube, optionally without covering them with a sleeve.

At 103, a distal portion of an endoscope is at least partially inserted into a designated lumen of the handle, so that the handle holds the distal portion of the add-on tube together with the distal portion of the endoscope. In some embodiments, a coupling element is provided inside the lumen of the handle, which attaches the distal end portion of the endoscope to a distal end portion of the add-on tube. Optionally, the coupling element is pre-attached to the distal end of the add-on tube, for example, the distal end of the add-on tube is mounted onto the coupling element. In an example, as further described herein, the coupling element comprises an expandable head which is expanded prior to insertion of the endoscope into the lumen of the handle, and fits onto to the endoscope distal end when the endoscope distal end is introduced inside. In some embodiments, the expandable head is configured to widen to receive endoscopes of various diameters, for example diameters of between 9-15 mm, 6-10 mm, 10-17 mm or intermediate, larger or smaller diameters.

Alternatively, in some embodiments, no coupling element is provided. Optionally, the handle is used only for covering an endoscope with a sleeve, without coupling an add-on tube to the endoscope.

In some embodiments, a distal end of the sleeve is attached to the expandable head, for example by an adhesive (e.g. glue, welding (e.g. RF welding), and/or other suitable attachment means).

At 105, at least a portion of the handle is pulled in the proximal direction over a least a portion of the remaining length of the endoscope and add-on tube which extend outside of the handle, pulling the thin elongate sleeve over the endoscope and add-on tube so that they are both covered by the sleeve. In some embodiments, the add-on tube and the endoscope extend side-by-side along the sleeve lumen. Optionally, the handle is pulled all the way to a proximal handle of the endoscope, covering the full length of the endoscope, optionally along with the add-on tube.

In some embodiments, pulling of the handle portion pulls on the sleeve to extend it from its axially compacted state to an extended, lengthened state.

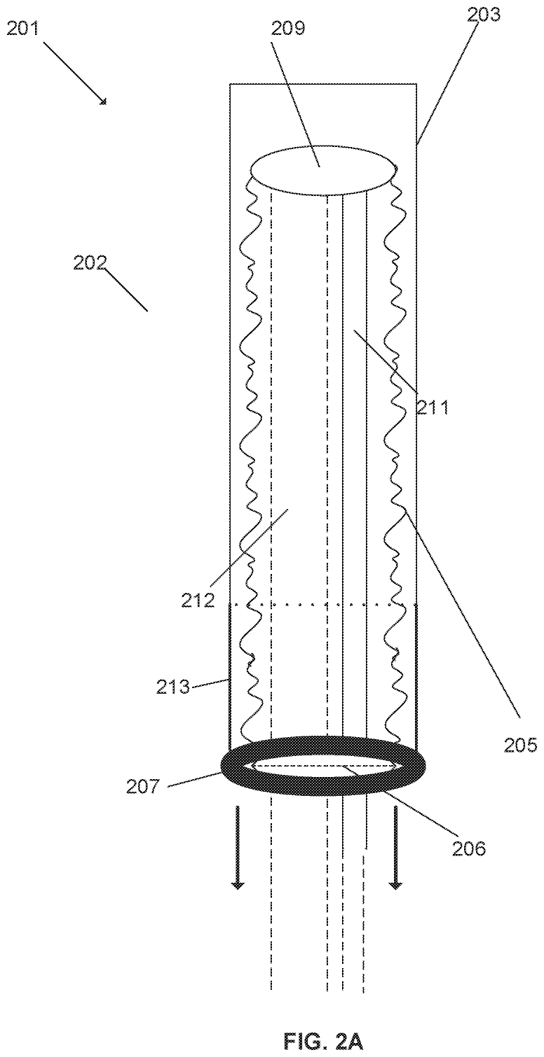

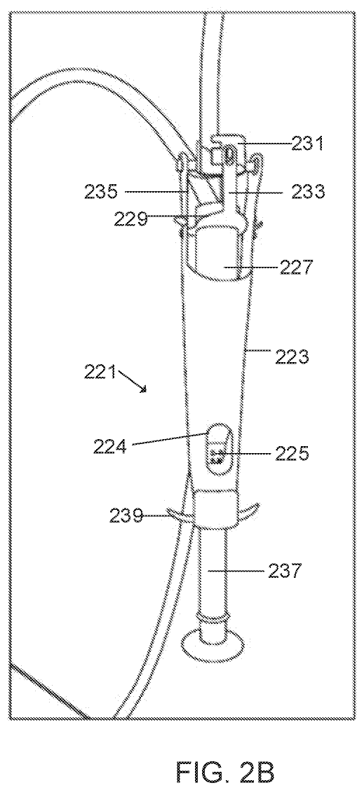

FIG. 2A schematically illustrates a loading handle pre-attached to an add-on tube and encasing a thin elongate sleeve for covering an endoscope optionally along with add-on tube, according to some embodiments.

In some embodiments, handle 201 includes a housing 202, where walls of the housing define a designated lumen 203 for receiving a distal end portion of an endoscope. In some embodiments, a thin elongate sleeve 205 is contained inside the designated lumen, optionally in an axially-collected state. In some embodiments, a proximal end of the sleeve comprises or is attached to a pulling element such as ring 207. In an example, the ring is attached to the proximal end of the sleeve by an adhesive (e.g. glue, welding (e.g. RF welding), or any other suitable attachment means).

In some embodiments, a distal end of the sleeve is attached to a coupling element 209 (e.g. an expandable head). In some embodiments, the coupling element 209 is fixedly held within the designated lumen of the handle. In an example, the expandable head is compressively held against the inner walls of the handle lumen by a plurality of arm extensions of the plunger which push radially outwardly. Optionally, after insertion of an endoscope distal end into the expandable head, the plunger (including the arm extension) is pulled away from the handle and the expandable head remains mounted on the distal end of the scope.

In some embodiments, a distal end of an add-tube 211 is pre-attached to the coupling element, for example, pre-mounted onto the coupling element.

In some embodiments, such as during preparation of an endoscope 212 for insertion into the body, a distal portion of the endoscope is inserted into the designated lumen of handle. The distal endoscope portion is advanced into attachment with the coupling element (for example, the coupling element defines a recess in which the distal end of the endoscope is fittingly received). Then, ring 207 is optionally detached from the handle and pulled in the proximal direction from the handle and along the long axis of the endoscope, stretching the sleeve from its compacted state and effectively lengthening the sleeve to cover at least a segment of the endoscope, optionally along with the add-on tube.

In some embodiments, ring 207 is sized to be large enough to axially slide over the endoscope, and optionally along the endoscope and add-on together. In an example, a diameter 206 of ring 207 is between 20-25 mm for example being large enough to encompass an endoscope having a diameter of 9-15 mm and an add-on tube having a diameter of 6-8 mm.

In some embodiments, ring 207 is coupled to the handle housing so that at least a portion 213 of the housing moves along with the ring. In some embodiments, a user manually grips portion 213 and slides it along the endoscope length to thereby slide the ring and dress the sleeve onto the endoscope.

It is noted that in some embodiments, a pullable element which is configured to pull on the end of the sleeve to dress the sleeve along the length of the endoscope is not necessarily ring-shaped, for example, shaped as a pullable tab, a rectangular frame, and/or other frame or structure which can be gripped or contacted by a user to be pulled back on.

FIG. 2B shows a loading handle pre-attached to an add-on tube and encasing a thin elongate sleeve for covering an endoscope and an add-on tube, according to some embodiments. In the exemplary handle structure shown, handle 221 comprises a body 223, for example, a conical or cylindrical body. Optionally, the body comprises a rounded (e.g. circular) cross section profile.

In some embodiments, an expandable head 225 is contained inside the body, for example fixedly positioned at a distal portion of the body. In some embodiments, body 223 comprises an opening or a window 224 through which the expandable head is visible.

In some embodiments, the expandable head comprises a basket or cage structure which is configured to radially expand in diameter, to receive a distal end of an endoscope inside. In some embodiments, the basket includes an external side pocket, and the distal end of the add-on tube is inserted and optionally mounted (e.g. glued) onto the side pocket.

In some embodiments, when multiple add-on tubes are coupled to the endoscope, the expandable head may include multiple pockets, optionally circumferentially arranged around the aperture in which the distal end of the endoscope is received, for accommodating the distal ends of the multiple add-on tubes.

In some embodiments, extending from the expandable head in a proximal direction is a gathered or bunched thin sleeve 227, attached at its proximal end to a ring 229.

In some embodiments, ring 229 is detachably connected to a gripper 231 which is configured proximally to the ring, and attached to the ring via one or more ring extensions 233. Optionally, extensions 233 connect between the gripper and the ring, for example via openable fasteners or other connectors configured for removable attachment. In some embodiments, the ring and the gripper are not connected to each other, as each can be independently pulled along the length of the endoscope.

In some embodiments, the ring and/or the gripper are removably connected to a plurality of arms 235 extending from a proximal end of the handle body 223. In use, the arms are detached from the ring and gripper to allow each of the gripper and ring to be pulled proximally away from the handle, separately or together.

Moving now to a distal portion of the handle, in some embodiments, a plunger 237 extends distally from the distal end of the handle body. In some embodiments, the plunger is positioned axially above the expandable head so that when the plunger is pushed proximally towards the body, a proximal portion of the plunger enters into the expandable head, pushing the walls of the head radially outwardly and expanding the head.

In some embodiments, the handle comprises a flange 239 located between the plunger and the body to enable resistively holding the handle by a user's hand while pushing on the plunger. In some embodiments, the flange is engaged by a user's hand when detaching the ring and/or gripper before pulling them proximally, to provide counter-resistance upon detachment.

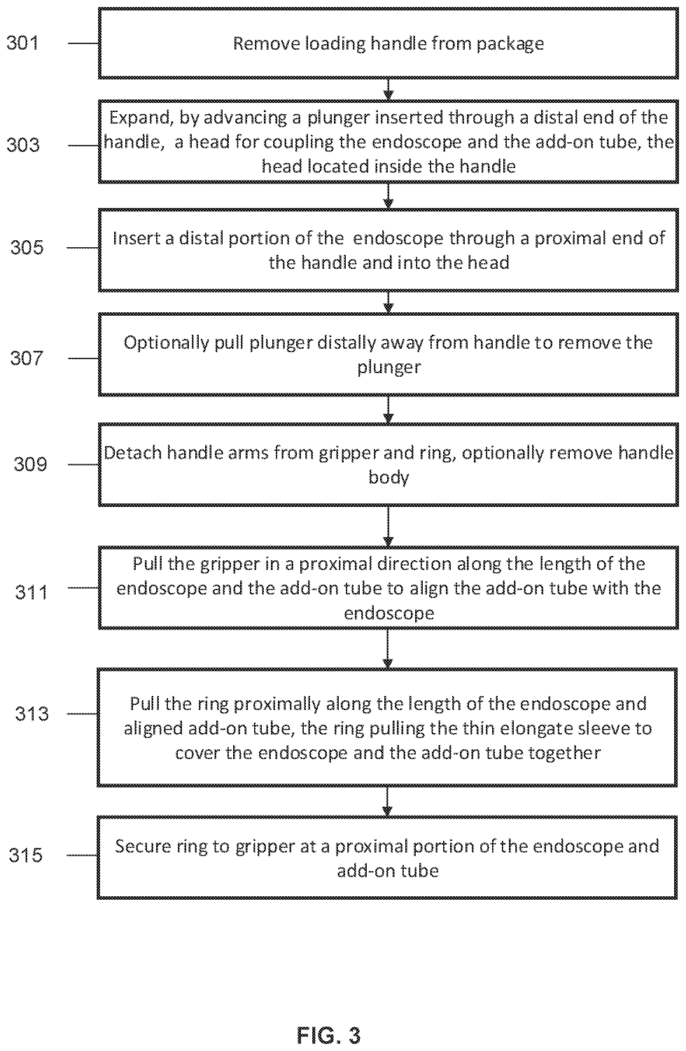

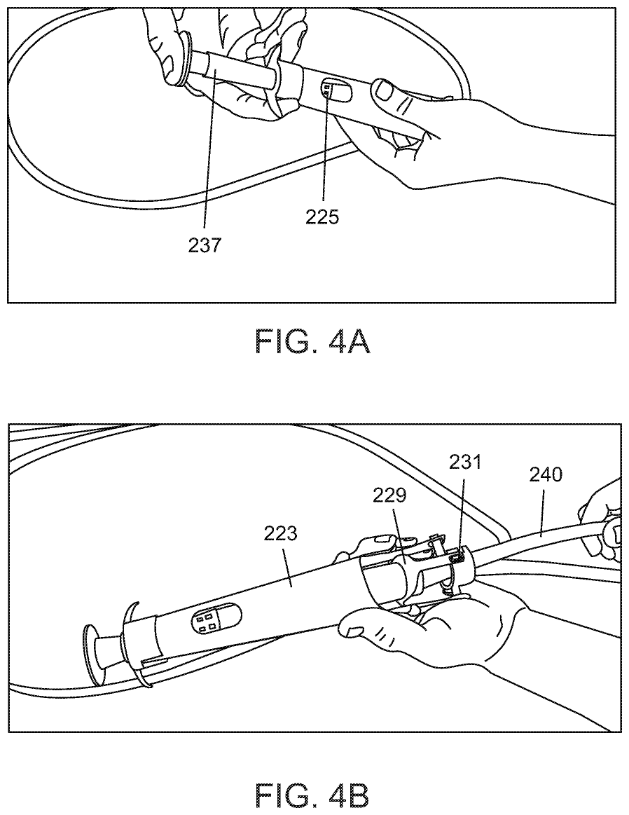

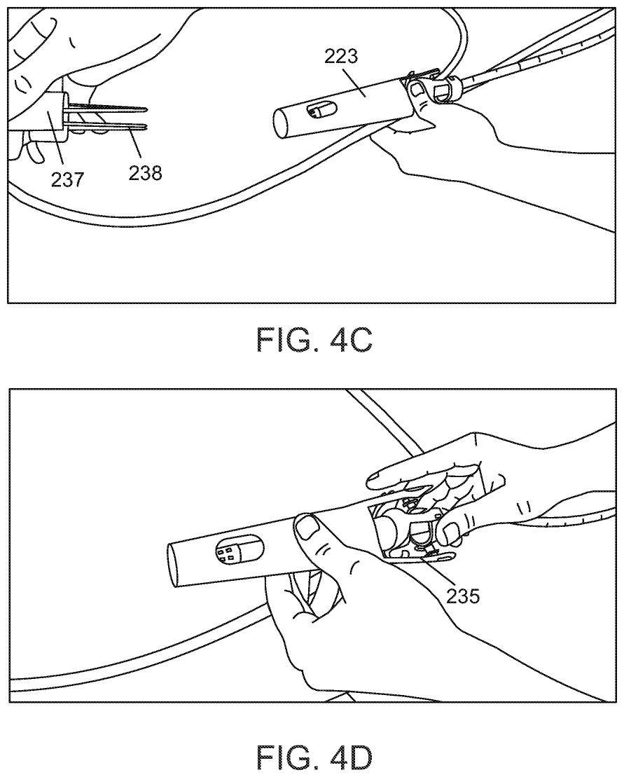

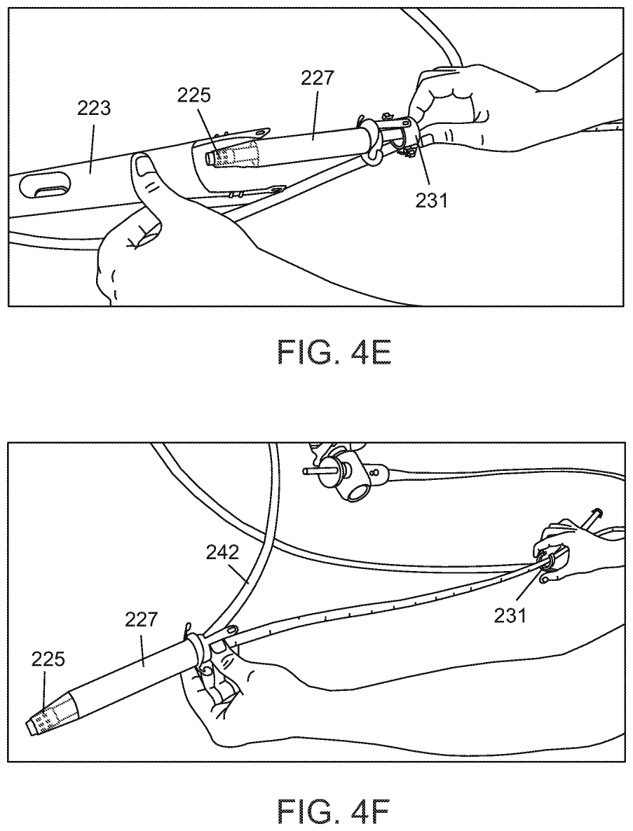

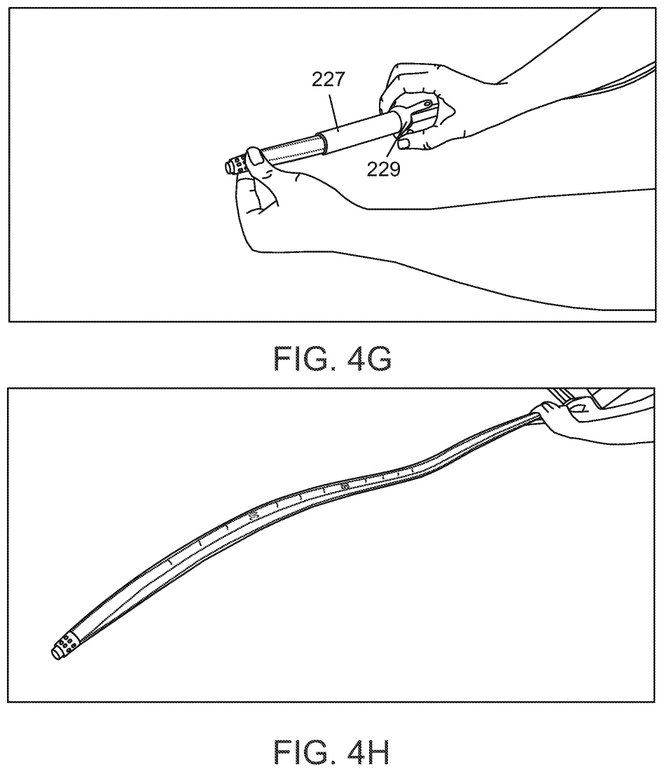

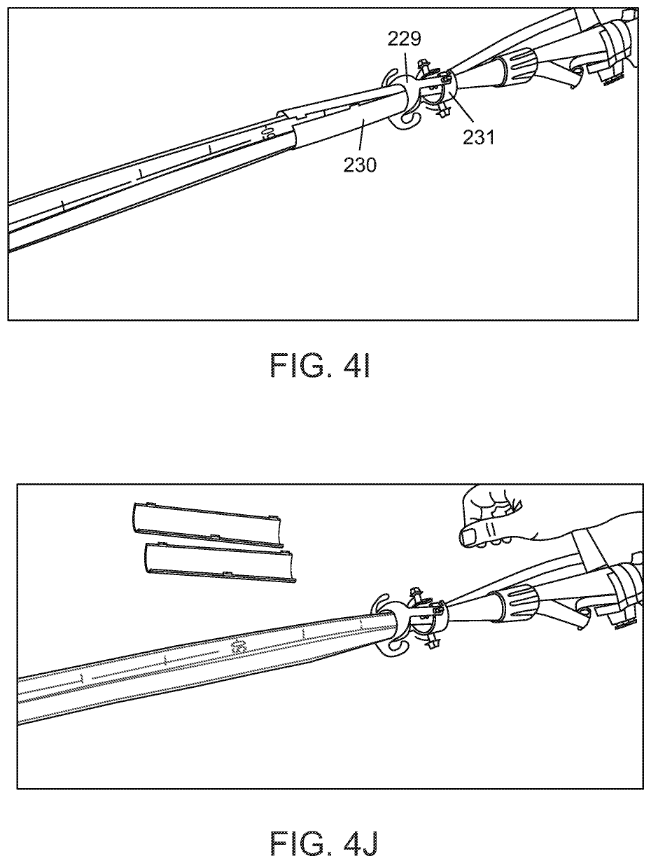

FIG. 3 is flowchart of a detailed method for covering an endoscope along with an add-on tube by an elongate thin sleeve using the loading handle of FIG. 2B, according to some embodiments. Steps of the method are demonstrated in the examples of FIGS. 4A-J.

In some embodiments, the method is carried out in preparation for insertion of an endoscope (e.g. a colonoscope) into the body.

At 301, in some embodiments, a loading handle for example as described herein is removed from its package.

At 303, in some embodiments, a coupling element such as an expandable head 225 is expanded to receive a distal end of an endoscope. In some embodiments, as demonstrated for example in FIG. 4A, a user holding the handle pushes the plunger 237 in a proximal direction. In some embodiments, a proximal end of the plunger comprises a plurality of arm extensions (visible in FIG. 4C, 238), which are pushed outwardly when the plunger is advanced, moving the side walls of the expandable head radially outwardly.

At 305, in some embodiments, as demonstrated for example in FIG. 4B, the user inserts a distal portion of an endoscope 240 into the lumen of the handle body 223. Optionally, the distal endoscope portion is advanced into the handle through an inner lumen of the ring 229 and of the gripper 231.

At 307, in some embodiments, as demonstrated for example in FIG. 4C, plunger 237 is pulled in a distal direction away from the handle and removed.

At 309, in some embodiments, as demonstrated for example in FIG. 4D, the handle arms 235 are detached from the gripper and the ring. In some embodiments, detaching is by pulling the arms radially outwardly to disconnect an attachment of an arm to the gripper and/or to the ring (in an example, the attachment is an interference fit attachment). In some embodiments, as demonstrated for example in FIG. 4E, once the handle arms are detached from the gripper and ring, handle body 223 is optionally removed as a whole from the endoscope, leaving the sleeve 227 (still in an axially-collected state) on a distal portion of the endoscope, with the add-on tube 242 (see FIG. 4F) held attached to the endoscope at their distal ends by the expandable head 225.

At 311, in some embodiments, as demonstrated for example in FIG. 4F, the gripper 231 is pulled (e.g. by sliding the gripper) in a proximal direction along the length of the endoscope, thereby approximating the add-on tube to the endoscope and aligning the add-on tube with respect to the endoscope. In some embodiments, when the gripper reaches a proximal position, it bands proximal portions of the endoscope and add-on together.

At 313, in some embodiments, as demonstrated for example in FIGs. G-H, the ring 229 is pulled (e.g. by sliding the ring) in a proximal direction, pulling on a proximal end of the sleeve 227 that is attached to it, until the sleeve covers the endoscope and the add-on tube that is aligned with it together along their length. Optionally, the ring is advanced along the full length of the endoscope to a proximal handle of the endoscope. In some embodiments, the ring remains attached to at least a portion of the handle body, which serves as a holder for the user to place their hand on when sliding the ring proximally. (See for example holder 230, visible in FIGS. 41-J.)

At 315, in some embodiments, as demonstrated for example in FIGS. 41-J, the ring that is now in a most proximal position is secured to the gripper, for example to prevent undesired movement of the ring back in a distal direction. Optionally, holder 230 is detached from the sleeve-covered assembly, for example by separating two or more portions of the holder apart. Optionally, the holder is disposed.

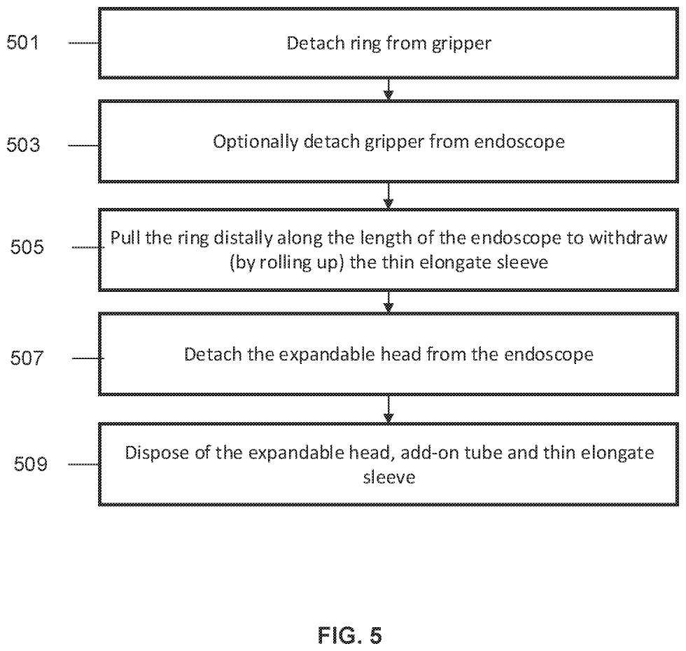

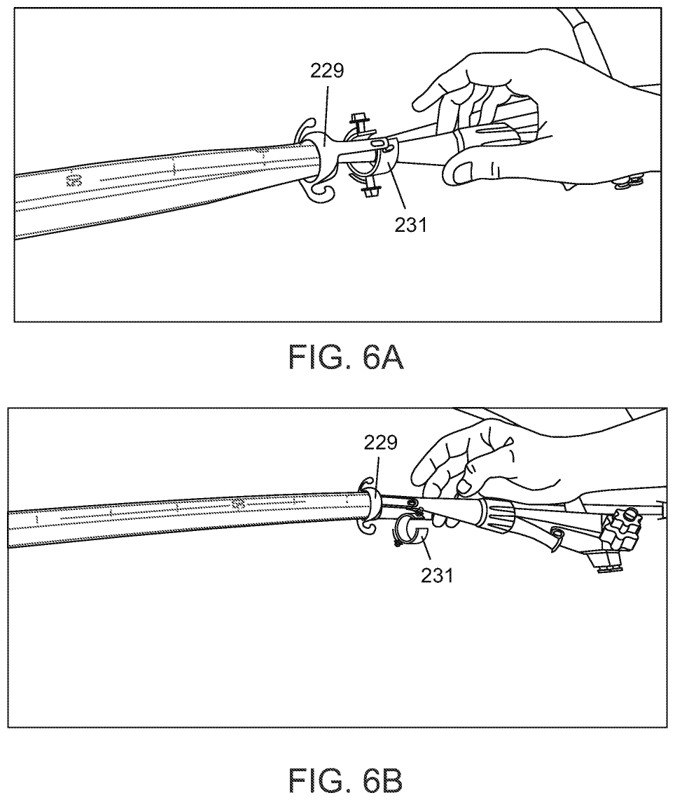

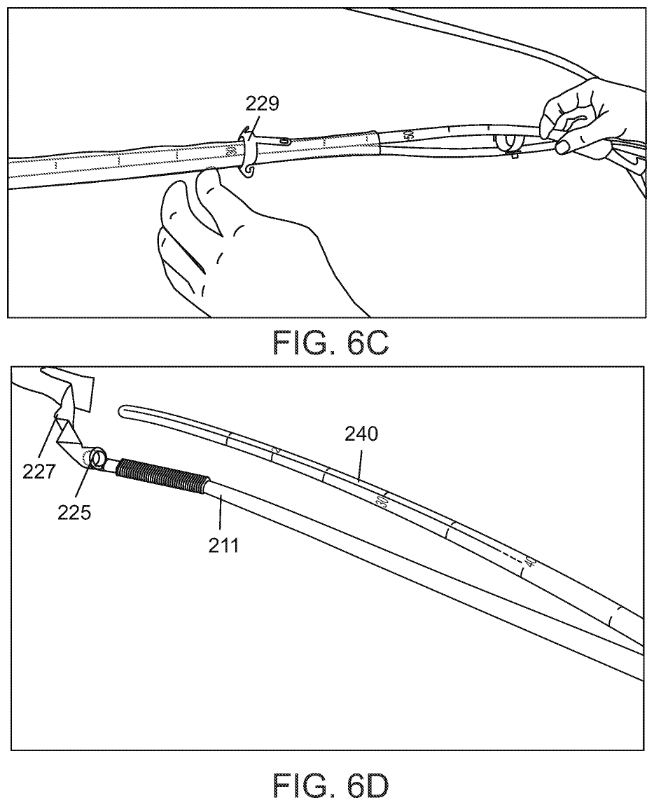

FIG. 5 is a flowchart of a method for removing the thin elongate sleeve and optionally separating the add-on tube from the endoscope following the endoscopic procedure, according to some embodiments. Steps of the method are demonstrated in the examples of FIGS. 6A-D.

At 501, in some embodiments, as demonstrated for example in FIG. 6A, the ring 229 is detached from the gripper 231. In some embodiments, disconnecting the ring from the gripper leaves the ring free to move (e.g. axially slide) along the endoscope.

At 503, optionally, as demonstrated for example in FIG. 6B, the gripper 231 is removed from the endoscope.

At 505, in some embodiments, as demonstrated for example in FIG. 6C, the ring 229 is moved (e.g. slid) in a distal direction along the length of the endoscope and add-on tube to withdraw the thin elongate sleeve by rolling the sleeve up.

At 507, in some embodiments, as demonstrated for example in FIG. 6D, after removing the sleeve 227, the distal end of the endoscope 240 is removed from the expandable head 225. Optionally, a distal end of the sleeve remains attached to the expandable head. Optionally, add-on tube 211 remains attached to the expandable head. Alternatively, the sleeve and/or add-on tube are detached from the head, for example by pulling them apart.

At 509, in some embodiments, the expandable head, add-on tube and sleeve are optionally disposed of. Optionally, the endoscope is cleaned or sterilized for additional use.

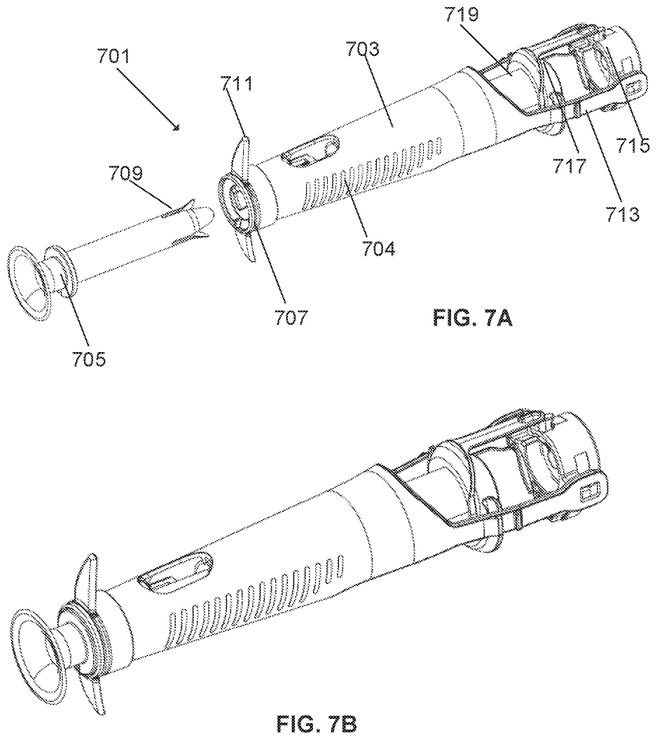

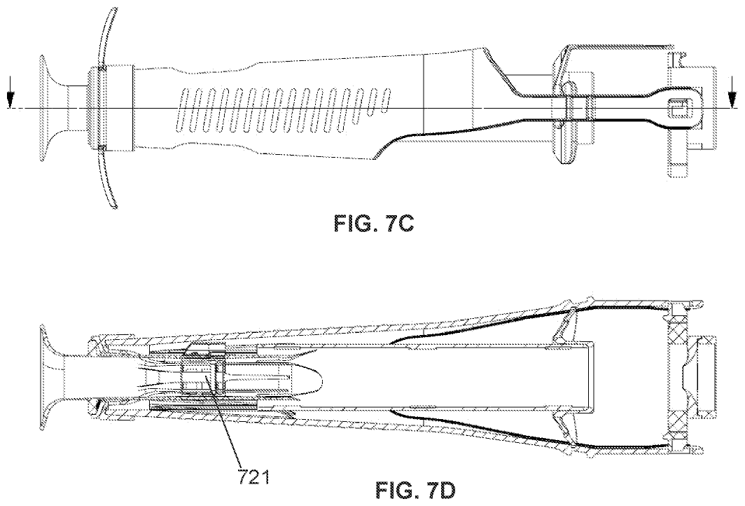

FIGS. 7A-D are various views of a loading handle 701, according to some embodiments. In some embodiments, the handle comprises: a handle body 703 (optionally, the handle body comprises one or more externally facing protrusions 704 or other surface irregularities for facilitating gripping of the handle by a user's hand); a plunger 705 positioned distally from a distal opening 707 of the body, the plunger comprising a plurality of arm extensions 709 shaped and configured to be pushed radially outwardly when the plunger is advanced in a proximal direction into the handle body (FIG. 7A shows the plunger and arm extensions when separated from the handle body; FIGS. 7B-D show the plunger at least partially pushed proximally into the lumen of the handle body); a flange 711 positioned around at least a portion of the distal end opening of the handle body; a plurality of arms 713 (e.g. 2, 3, 4, 6 or intermediate, larger or smaller number) extending proximally from a proximal end of the handle body, the arms connected by a removable coupling to a gripper 715 and or ring 717; a thin elongate sleeve 719 (optionally provided pre-loaded in the handle), the sleeve being attached to the ring 717 at its proximal end and to coupling element such an expandable head 721 (shown in the cross section view of FIG. 7D) at its distal end, where the expandable head is accommodated inside the handle body.

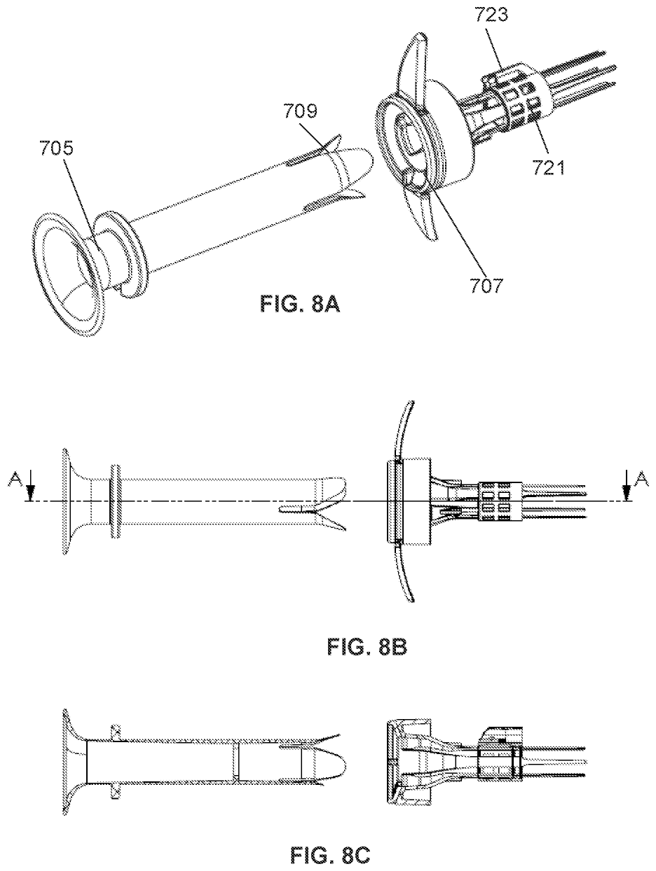

FIGs. 8A-C are various views of the plunger 705 and the inner distal portion of the loading handle which includes a coupling element (e.g. an expandable head 721) for coupling the add-on tube to the endoscope, according to some embodiments.

In some embodiments, when plunger 705 is inserted through the distal opening 707 of the body, arm extensions 709 enter the expandable head through a distal opening of the head and push on the walls of the expandable head to widen it for receipt of an endoscope distal end within. In some embodiments, each of the arm extensions splits into additional narrower branches. In some embodiments, some of the arm extensions are longer than others. A potential advantage of the expandable arm extensions may include: compressively supporting the expandable head inside the lumen of the handle, expanding the expandable head when being advanced distally by the plunger, and optionally facilitating release of the plunger when it is pulled proximally away from the handle.

In some embodiments, a distal end of an add-on tube (not shown) is attached (e.g. mounted into) a side pocket 723 of the expandable head.

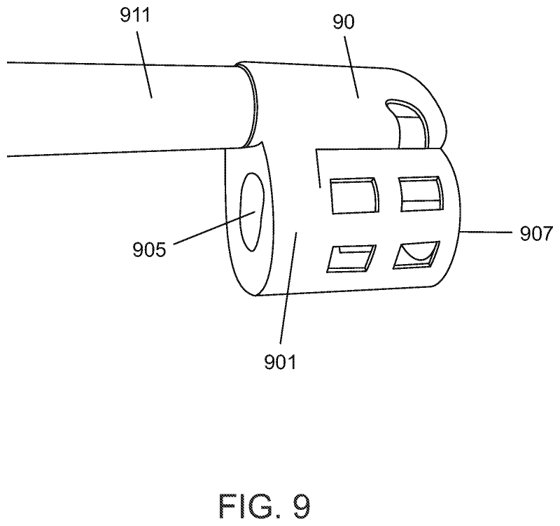

FIG. 9 is an image of a coupling element for coupling a distal end of an endoscope to a distal end of an add-on tube, according to some embodiments. In some embodiments, the coupling element is comprised of an expandable head 901 having a basket or cage-like structure. In some embodiments, the expandable head includes a proximal opening 905 for insertion of an endoscope distal end through. In some embodiments, the expandable head includes a distal opening 907 shaped and sized for exposing of a distal face of the endoscope.

In some embodiments, the expandable head includes a fixture such as a side pocket 90 in which a distal end of an add-on tube 911 is received. Optionally, the add on tube is mounted (e.g. glued) onto the inner walls of the pocket. In some embodiments, the pocket is open at its distal end. In some embodiments, the expandable head is formed of a flexible material such as Polyurethane (PU), PVC, TPE, TPU, Silicone which enables the walls of the head to widen in a radially outwards direction, for example for closely fitting onto endoscopes of various diameters.

In some embodiments, the expandable head maintains the add-on tube attached to the endoscope received therein. In some embodiments, the expandable head remains mounted onto the distal end of the endoscope throughout the procedure. In some embodiments, the expandable head is shaped and sized so that it does not interfere with functions carried out through the endoscope and/or through the add-on tube, for example: visualization via the endoscope camera, irrigation/suction via the add-on tube, insertion and manipulation of tools through the endoscope working channel, irrigation/suction via the endoscope working channel, and as such.

In some embodiments, no coupling element (e.g. no expandable head) is provided. In an example, the distal end of the sleeve is attached directly to the handle body.

In some embodiments, the distal end of the sleeve is closed and is made of a transparent material so that the sleeve does not interfere with visualization via the endoscope.

In some embodiments, no handle body is provided.

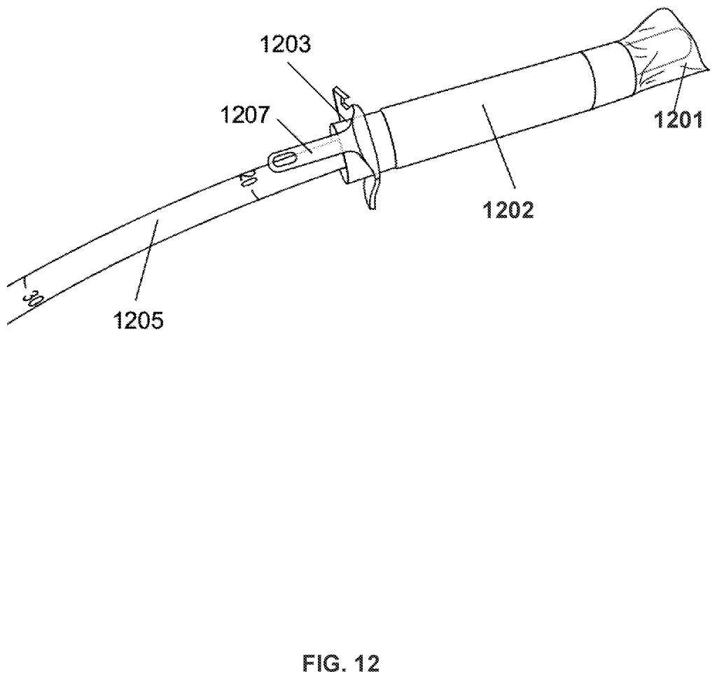

In some embodiments, as shown for example in FIG. 12 a sleeve 1201 is provided with a pullable element, e.g. a ring 1203, attached to the sleeve proximal end. In some embodiments, the sleeve is crimped (e.g. axially collected) on a handle body 1202 (for example, a cylindrical portion). In some embodiments, in use, an endoscope 1205 is inserted into the sleeve, optionally through the ring and handle body 1202, and advanced distally until the endoscope head reaches the closed distal end of the sleeve. Then, the ring is pulled in a proximal direction along the length of the endoscope, thereby pulling on the proximal end of the sleeve and uncrimping the sleeve. Optionally, the ring comprises a tab 1207 for facilitating grasping by the user. In some embodiments, in use, pulling the ring in a proximal direction along the length of the endoscope gradually uncrimps the axially collected sleeve from a distal end of the sleeve, stretching the opened sleeve along the endoscope.

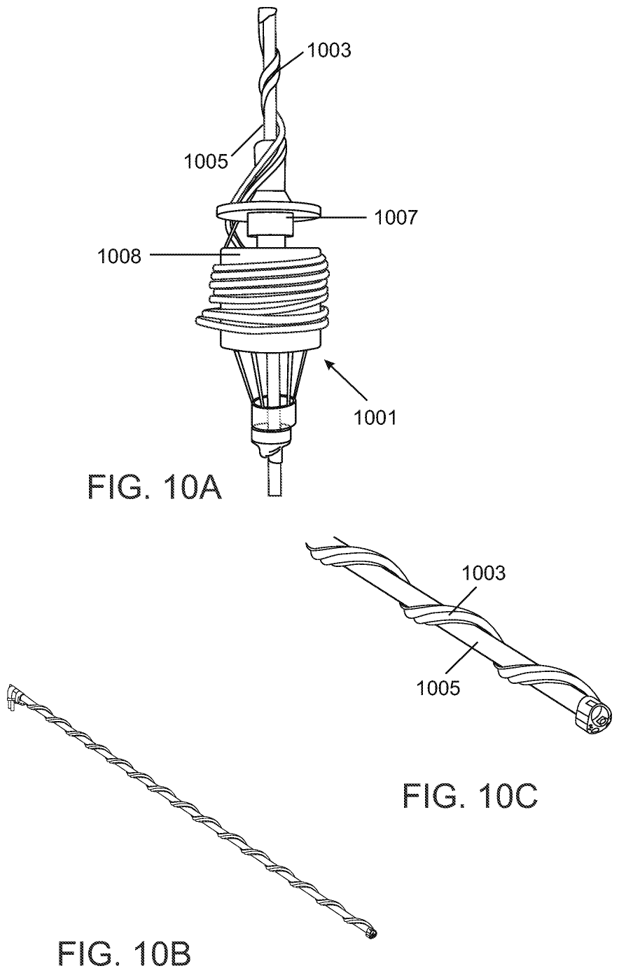

FIGS. 10A-C show a handle 1001 for spirally coupling an add-on tube to an endoscope (FIG. 10A), and schematic views of an add-on tube spirally coupled to an endoscope (FIGS. 10B-C), according to some embodiments.

In some embodiments, handle 1001 is configured for coupling an add-on tube 1003 to an endoscope 1005. In some embodiments, the handle comprises a frame 1007 on which the add-on tube is mounted, for example, the add-on tube is axially rolled on the frame, such as on a cylindrical portion 1008 of the frame.

In some embodiments, frame 1007 defines an inner lumen or a recess in which endoscope 1005 is received. In some embodiments, a proximal portion of the frame, or optionally the frame as a whole, is configured to slide (for example by manual pulling by a user) along the length of the endoscope, thereby stretching the add-on tube relative to the endoscope. In some embodiments, the add-on tube is disposed in a spiral manner along the length of the endoscope, as shown for example in FIGS. 10B, 10C. In an example, in use, the rolled add-on tube is pulled in a proximal direction along the endoscope by the frame, and sections of the add-on tube are gradually released by the frame to be spirally mounted on the endoscope.

In some embodiments, at least a portion of the frame, such as cylindrical portion 1008, is configured to rotate during pulling of the frame in the proximal direction, providing for winding the add-on tube around the endoscope.

In some embodiments, the frame comprises or is coupled to an expandable head and optionally to plunger and arm extensions for example as described herein, for coupling a distal head of the endoscope to a distal end of the add-on tube, and holding them together.

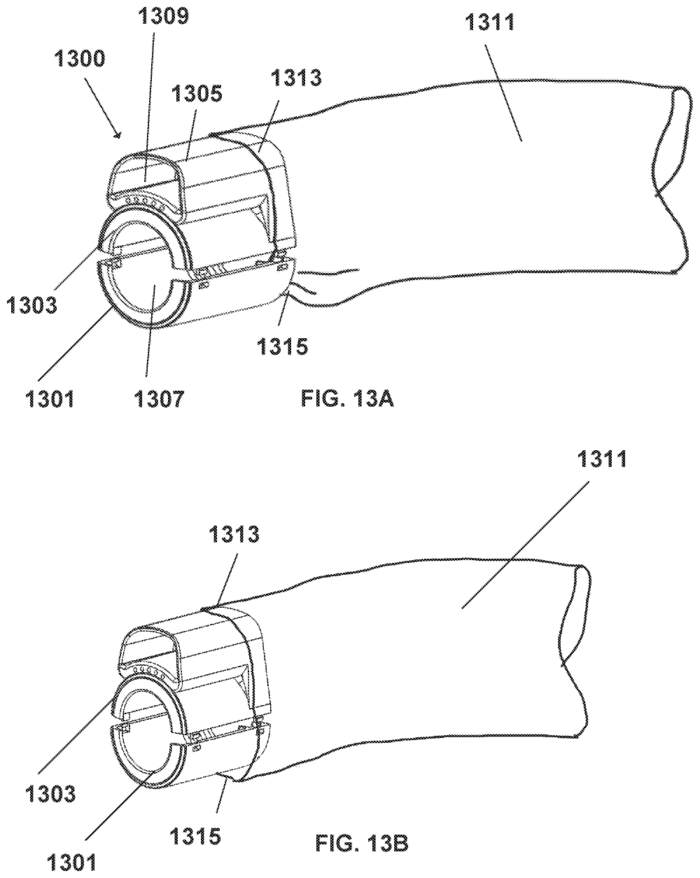

FIGS. 13A-B are examples of a snap-fit head for coupling an add-on tube to an endoscope, according to some embodiments.

In some embodiments, as shown for example in FIG. 13A, a head 1300 for positioning on a distal end of an endoscope is comprised of a plurality of attachable portions, e.g. portions 1301, 1303, which are configured to attach to each other and optionally interlock with each other, for example via a snap-fit coupling, an interference fit coupling, latches, and the like. In some embodiments, the multiple portions are shaped to define, when attached, an inner lumen 1307 in which the endoscope distal end is received.

In some embodiments, head 1300 comprises a side pocket 1305 shaped and sized to receive a distal end of an add-on tube therein. In some embodiments, pocket 1305 is mounted externally to the one or more portions 1301, 1303 of the head. In some embodiments, an inner lumen 1309 of pocket 1305 has a cross-section profile which is shaped to match an add-on tube profile, for example, a trapezoidal profile as shown, a circular profile, or any other profile.

In some embodiments, as shown for example in FIG. 13A, at least a portion of a sleeve 1311 is attached to at least a portion of the head. In an example, a first half of the sleeve circumference 1313 is attached (e.g. glued or welded) to the first portion 1303 of the head, and a second half 1315 of the sleeve circumference is pinched between the second portion of the head 1301 and the body of the endoscope (when the endoscope distal end is inserted into the inner lumen of the head). In another option, shown for example in FIG. 13B, a portion of the sleeve (e.g. a first half 1313 of the sleeve circumference) is attached to the head (e.g. glued or welded externally to the pocket), while a different portion of the sleeve (e.g. a second half 1315 of the sleeve circumference) remains hanging open. In some embodiments, the rest of the sleeve length is optionally crimped (e.g. axially folded) and can be pulled on to cover the endoscope and add-on tube together along their length, for example as described elsewhere in this application.

It is noted that various head configurations for holding together a distal end portion of an endoscope and a distal end of portion of an add-on tube are contemplated in this application, for example, a flexible head (e.g. a flexible cap), a rigid head (e.g. a rigid cap). In some embodiments, the head includes a partition defining at least two inner axial lumens—a first lumen for receiving the endoscope distal end portion, and a second lumen for receiving the add-on tube distal end portion. In some embodiments, the head includes cylindrical portion for receipt of the endoscope, and a pocket portion mounted externally to the cylindrical portion for receipt of the add-on tube.

In some embodiments, the head is placed onto the endoscope distal end by fitting the head onto the endoscope body. Additionally or alternatively, the endoscope is inserted into the lumen of the head and optionally pushed in.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A handle for at least partially covering an endoscope with a protective thin sleeve, the handle comprising:
a handle body shaped and sized to receive a distal portion of an endoscope therein;
a detachable portion at a proximal end of said handle body, said detachable portion configured to detach from said handle body and to be moved proximally along at least a portion of a length of the endoscope received within said handle body; and
a thin elongate protective sleeve axially compacted within said handle body, a proximal end of said sleeve coupled to said detachable portion, wherein movement of said detachable portion proximally along said endoscope pulls on said proximal end of said sleeve and extends said sleeve in length from its axially compacted state to a lengthened state, thereby covering said at least a portion of the length of said endoscope.

2. The handle according to claim 1, wherein said detachable portion comprises a ring which is separable from said handle body, said ring sized to circumferentially surround an endoscope received within said handle body.

3. The handle according to claim 1, wherein said handle body accommodates an expandable head sized to receive a distal end of the endoscope therein, said expandable head coupled to a distal end of said sleeve.

4. The handle according to claim 3, wherein said expandable head is coupled to a distal end of an add-on tube to be inserted along with the endoscope into the body, said expandable head configured to hold a distal end of the endoscope together with said distal end of said add-on tube.

5. The handle according to claim 4, further comprising a plunger positioned distally to said expandable head, the plunger comprising arm extensions shaped and configured to fit into said expandable head to widen said expandable head for receipt of an endoscope distal end therein.

6. The handle according to claim 3, wherein said expandable head is elastically expanded to a diameter of between 10-20 mm to fit endoscopes having a diameter of between 9-15 mm.

7. (canceled)

8. The handle according to claim 1, wherein said detachable portion further comprises a gripper which is separable from said handle body, the gripper circumferentially surrounding said add-on tube and an endoscope received within the handle, the gripper configured to slide proximally to align and approximate said add-on tube to said endoscope.

9. The handle according to claim 1, wherein said sleeve is formed of a flexible material.

10. The handle according to claim 1, wherein said sleeve is transparent at least at a distal end face of said sleeve.

11. A method of covering an endoscope with a protective thin sleeve, comprising:
providing a loading handle containing an axially-collected protective thin sleeve therein;
inserting a distal end portion of an endoscope into a lumen of said loading handle such that said distal end portion of said endoscope is received within said sleeve;
detaching a proximal portion of said handle which is coupled to a proximal end of said sleeve; and
pulling said proximal portion of said handle along a length of said endoscope to cover said endoscope with said sleeve.

12. The method according to claim 11, further comprising, following an endoscopy procedure, removing said sleeve from said endoscope by pulling said proximal portion of said handle in a proximal direction along the length of said endoscope, thereby rolling up said sleeve.

13. The method according to claim 11, comprising coupling, at said handle, a distal end of an add-on tube to said distal end portion of said endoscope; wherein said pulling comprises pulling said proximal portion of said handle over said add-on tube and said endoscope to cover them together by said sleeve.

14. The method according to claim 13, wherein said coupling comprises positioning said distal end portion of said endoscope within a coupling element accommodated inside said lumen of said handle, said coupling element attached to said distal end of said add-on tube.

15. The method according to claim 11, wherein said pulling of said proximal portion of said handle is configured to align and approximate said add-tube to said endoscope along their length.

16. The method according to claim 11, comprising disposing said handle, sleeve and add-on tube following use.

17-18. (canceled)

19. The handle according to claim 1,
wherein said handle body accommodates a coupling element configured to couple the distal end of the endoscope to a distal end of an add-on tube.

20. (canceled)

21. A handle for spirally mounting an add-on tube on an endoscope, the handle comprising: a frame comprising:
a recess in which at least a portion of an endoscope is received;
a cylindrical portion on which an add-on tube is axially rolled;
at least a portion of the frame configured to slide along a length of the endoscope and release the rolled add-on tube such that the add-on tube is spirally disposed about the endoscope.

22-23. (canceled)

24. The handle according to claim 19, said coupling element comprising:
a plurality of portions shaped to define, when attached to each other, an inner lumen shaped and sized for fittingly receiving the endoscope distal end therein, said plurality of portions configured to interlock with each other; and
a pocket shaped and sized for receiving the add-on tube distal end therein, the pocket being externally mounted on at least one of said plurality of portions and located radially outwardly relative to said inner lumen.

25. (canceled)

26. The handle according to claim 3, wherein said
expandable head includes a circumferential wall which defines an inner recess, said circumferential wall being expandable in a radially outwards direction;
a plunger shaped to compressively fit inside at least a portion of said inner recess to thereby push said circumferential wall radially outwardly to provide for insertion of the endoscope distal end into said inner recess.

27. The handle according to claim 26, wherein said plunger comprises a plurality of arm extensions configured for insertion into said inner recess, wherein said arm extensions are pushed radially outwardly against said circumferential wall of said head when said plunger is advanced into said inner recess.
